(19) United States
(12) Patent Application Publication
(10) Pub. No.: US 2023/0347006 A1
(43) Pub. Date: Nov. 2, 2023

(54) STERILIZATION ATOMIZATION DEVICE

(71) Applicants: Guanghui ZU, Shenzhen (CN); SHENZHEN JINGXINTAI HOUSEWARE CO., LTD., Shenzhen (CN)

(72) Inventors: JINSHAN ZOU, Shenzhen (CN); Guanghui ZU, Shenzhen (CN); Aiqun LIN, Shenzhen (CN)

(21) Appl. No.: 17/945,761

(22) Filed: Sep. 15, 2022

(30) Foreign Application Priority Data
Apr. 29, 2022 (CN) 202210468942.7

(51) Int. Cl. A61L 9/14; B05B 17/06

(52) U.S. Cl. CPC A61L 9/14; B05B 17/0661; A61L 2209/11; H02J 7/0063

(57) ABSTRACT

The present application relates to the technical field of atomization devices, and provides a sterilization atomization device, including a housing provided with a first liquid storage cavity; an electrolysis module, disposed in the first liquid storage cavity and configured for electrolyzing the electrolyte accommodated in the first liquid storage cavity; an atomization module; and a power module. The atomization module is disposed in the first liquid storage cavity and configured for atomizing the electrolyte accommodated in the first liquid storage cavity into an atomized airflow, the atomized airflow flows out of the housing through at least the first liquid storage cavity; and the power module is configured for supplying power to the electrolysis module and the atomization module independently at the same time.

### CROSS REFERENCE TO RELATED APPLICATION

The present application claims priority of Chinese Patent Application, with Application No. 202210468942.7, filed on Apr. 29, 2022, the content of which is incorporated herein by reference.

### TECHNICAL FIELD

The present application relates to the technical field of atomization devices, and more particularly to a sterilization atomization device.

### BACKGROUND

At present, the sterilization atomization devices on the market generally use UV sterilization or heat sterilization to achieve the sterilization function. The above sterilization atomization devices can only kill bacteria in water, but cannot kill bacteria in the air, and the sterilization rate can only reach about 60%, and the sterilization effect is poor.

In order to improve the sterilization effect of the sterilization atomization device, in the related art, there is a sterilization atomization device sterilized by electrolysis. The above-mentioned sterilization atomization device sterilized by electrolysis includes an electrolysis module and an atomization module, the electrolysis module is used to electrolyze the electrolyte to generate sterilization substances in the electrolyte, and the sterilization substances can kill bacteria in the electrolyte, and the atomization module is used to atomize the electrolyte, the atomized electrolyte can kill bacteria in the air since the electrolyte contains sterilizing substances, and the sterilization effect is better. However, in the process of using the above-mentioned sterilization atomization device sterilized by electrolysis, the electrolytic reaction between the electrodes of the electrolysis module and the metal materials of the atomization module (for example, the silver paste layers used for electrical connection with the power module) is easy to occur, which leads to corrosion of the metal materials on the atomization module, which in turn affects the service life of the atomization device.

### SUMMARY

An object of the present application is to provide a sterilization atomization device, to solve the technical problem that in the related art, the electrolytic reaction between the electrodes of the electrolysis module and the metal materials of the atomization module is easy to occur, which leads to corrosion of the metal materials on the atomization module, which in turn affects the service life of the atomization device.

In order to achieve above object, the technical solution adopted by the present application is to provide a sterilization atomization device, which includes:
a housing, provided with a first liquid storage cavity configured for accommodating an electrolyte;
an electrolysis module, disposed in the first liquid storage cavity and configured for electrolyzing the electrolyte accommodated in the first liquid storage cavity;
an atomization module, disposed in the first liquid storage cavity and configured for atomizing the electrolyte accommodated in the first liquid storage cavity into an atomized airflow, the atomized airflow flows out of the housing through at least the first liquid storage cavity; and
a power module, configured for supplying power to the electrolysis module and the atomization module independently at the same time.

In one embodiment, the power module includes a first input circuit and a first conversion circuit that are connected to each other, and the first conversion circuit is configured for converting a first input voltage of the first input circuit into a first output voltage and a second output voltage that are mutually isolated, and the first output voltage and the second output voltage are respectively transmitted to the atomization module and the electrolysis module.

In one embodiment, the power module includes a storage battery, a second input circuit and a second conversion circuit, and the second conversion circuit is configured for converting a second input voltage of the second input circuit into a third output voltage, and the third output voltage is transmitted to the atomization module, and the electrolysis module is powered by the storage battery.

In one embodiment, the electrolysis module includes a first connection assembly, a second connection assembly, a first electrode sheet, an insulating sheet and, a second electrode sheet; the first electrode sheet, the insulating sheet and the second electrode sheet are stacked in sequence, the first connection assembly is configured for fixing the first electrode sheet on a cavity wall of the first liquid storage cavity and for connecting the first electrode sheet to the power module; and the second connection assembly is configured for fixing the second electrode sheet on the cavity wall of the first liquid storage cavity and for connecting the second electrode sheet to the power module.

In one embodiment, at least one of the first electrode sheet and the second electrode sheet includes a titanium metal layer and a composite coating layer coated on a surface of the titanium metal layer, and the composite coating layer is a ruthenium-iridium composite material layer, a ruthenium-platinum composite material layer or a platinum-iridium composite material layer.

In one embodiment, the atomization module includes a piezoelectric ceramic sheet and two silver paste layers respectively disposed on an upper surface and a lower surface of the piezoelectric ceramic sheet, and the two silver paste layers are configured for electrically connecting with the power module.

In one embodiment, the sterilization atomization device further includes a switch module, and the housing is further provided with a second liquid storage cavity configured for accommodating the electrolyte, the switch module configured for communicating the second liquid storage cavity and the first liquid storage cavity when a liquid level of the first liquid storage cavity is lower than a first preset value, such that the second liquid storage cavity supplies the electrolyte to the first liquid storage cavity, and the switch module is configured for isolating the second liquid storage cavity from the first liquid storage cavity when the liquid level of the first liquid storage cavity is higher than a second preset value, such that the second liquid storage cavity stops supplying the electrolyte to the first liquid storage cavity, and the first preset value is less than or equal to the second preset value.

In one embodiment, the housing includes a liquid storage tank and a base, the liquid storage tank is detachably mounted on the base, the second liquid storage cavity is arranged in the liquid storage tank, and a bottom portion of the liquid storage tank and a top portion of the base are enclosed to form the first liquid storage cavity, the bottom portion of the liquid storage tank is provided with a water outlet connecting the second liquid storage cavity and the first liquid storage cavity, the first preset value is equal to the second preset value, and the switch module includes a normally shut switch and a ball float type trigger, the normally shut switch is arranged at the water outlet, and the ball float type trigger is arranged at the top portion of the base and is configured for triggering the normally shut switch to open when the liquid level is lower than the first preset value.

In one embodiment, the housing further includes an air inlet cavity and an air inlet communicated with the air inlet cavity, and the air inlet cavity is arranged below the first liquid storage cavity; the sterilization atomization device further includes a mist guiding tube and a fan module, a bottom portion of the mist guiding tube is communicated with the air inlet cavity, a middle portion of the mist guiding tube is communicated with the top portion of the first liquid storage cavity, and a top portion of the mist guiding tube is communicated with an outside of the housing; the fan module is arranged at the bottom portion of the mist guiding tube, and the fan module is configured for driving an air entering the mist guiding tube through the air inlet and the air inlet cavity in turn to carry the atomized airflow from the top portion of the first liquid storage cavity to an outside of the housing.

In one embodiment, a region corresponding to the atomization module on a top wall of the first liquid storage cavity is provided with a mounting hole configured for communicating with the middle portion of the mist guiding tube, the sterilization atomization device further includes a noise reduction module, and the noise reduction module is mounted at the mounting hole.

In one embodiment, the sterilization atomization device further includes a control module, the control module is electrically connected with the electrolysis module and the atomization modules, respectively; the housing further includes a control cavity, and the control module is arranged in the control cavity.

The beneficial effect of the sterilization atomization device provided by the present application is that, compared with the related art, in the sterilization atomization device provided by the present application, the power module can simultaneously supply power to the electrolysis module and the atomization module independently, that is, the power module can simultaneously supply power for the electrolysis module and atomization module, and the circuit where the electrolysis module is located and the circuit where the atomization module is located do not share the same ground. In this way, it can not only ensure that the electrolysis module and the atomization module can work at the same time, ensure the sterilization effect, but also ensure that when the electrodes of the electrolysis module (such as the electrodes including the titanium metal layer and the platinum metal layer coated on the surface of the titanium metal layer) and the metal materials on the atomization module (such as the silver paste layer for electrical connection with the power module) are exposed in the electrolyte, no electrolytic reaction will occur between the electrodes of the electrolysis module and the metal materials on the atomization module, and the metal materials on the atomization module will not be corroded, which is beneficial to improve the service life of the sterilization atomization device.

### BRIEF DESCRIPTION OF THE DRAWINGS

In order to explain the embodiments of the present application more clearly, a brief introduction regarding the accompanying drawings that need to be used for describing the embodiments of the present application or the prior art is given below; it is obvious that the accompanying drawings described as follows are only some embodiments of the present application, for those skilled in the art, other drawings can also be obtained according to the current drawings on the premise of paying no creative labor.

FIG. 1 is a first partial structural schematic view of a sterilization atomization device provided by an embodiment of the present application;

FIG. 2 is a structural schematic view of a power module provided by an embodiment of the present application;

FIG. 3 is a structural schematic view of another power module provided by an embodiment of the present application;

FIG. 4 is a second partial structural schematic view of a sterilization atomization device provided by an embodiment of the present application;

FIG. 5 is an enlarged view of part A of the sterilization atomization device shown in FIG. 4;

FIG. 6 is a third partial structural schematic view of a sterilization atomization device provided by an embodiment of the present application;

FIG. 7 is an enlarged view of part B of the sterilization atomization device shown in FIG. 6;

FIG. 8 is a first explosive schematic view of a sterilization atomization device provided by an embodiment of the present application; and

FIG. 9 is a second explosive schematic view of a sterilization atomization device provided by an embodiment of the present application.

In the drawings, the reference signs are listed:
100-housing; 110-first liquid storage cavity; 120-second liquid storage cavity; 130-liquid storage tank; 131-water outlet; 132-liquid storage tank body; 133-tank cover; 134-air outlet plate; 135 -fixing member; 140-base; 141-base body; 142-bottom cover; 150-air inlet cavity; 160-air inlet; 170-air outlet; 180-mounting hole; 190-control cavity;
200-electrolysis module; 210-first connection assembly; 211-first bolt; 212-first nut; 220-second connection assembly; 221-second bolt; 222-second nut; 230-first electrode sheet; 240-insulating sheet; 241-first convex ring; 242-second convex ring; 250-second electrode sheet;
300-atomization module;
400-power module; 410-first input circuit; 420-first conversion circuit; 430-storage battery; 440-second input circuit; 450-second conversion circuit;
500-switch module; 510-normally shut switch; 511-valve cover; 512-elastic member; 513-valve rod; 514-cover plate; 520-ball float type trigger; 521-connecting rod; 522-ball float; 523-ejector rod;
600-mist guiding tube; 610-upper guiding tube; 620-lower guiding tube;
700-fan module;
800-noise reduction module; 810-drainage cover; 811-first through hole; 812-second through hole;
900-control module;
m-first liquid level line; and
n-second liquid level line.

### DETAILED DESCRIPTION OF EMBODIMENTS

Herein, embodiments of the present application are described in detail, and examples of the embodiment are illustrated in the accompanying figures; wherein, an always unchanged reference number or similar reference numbers represent(s) identical or similar components or components having identical or similar functionalities. The embodiment described below with reference to the accompanying figures is illustrative and intended to illustrate the present application, but should not be considered as any limitation to the present application.

In the description of the present application, it needs to be understood that, directions or location relationships indicated by terms such as “length”, “width”, “up”, “down”, “front”, “rear”, “left”, “right”, “vertical”, “horizontal”, “top”, “bottom”, “inside”, “outside”, and so on are the directions or location relationships shown in the accompanying figures, which are only intended to describe the present application conveniently and simplify the description, but not to indicate or imply that an indicated device or component must have specific locations or be constructed and manipulated according to specific locations; therefore, these terms shouldn’t be considered as any limitation to the present application.

In addition, terms “the first” and “the second” are only used in describe purposes, and should not be considered as indicating or implying any relative importance, or impliedly indicating the number of indicated technical features. As such, technical feature(s) restricted by “the first” or “the second” can explicitly or impliedly comprise one or more such technical feature(s). In the description of the present application, “a plurality of” means two or more, unless there is additional explicit and specific limitation.

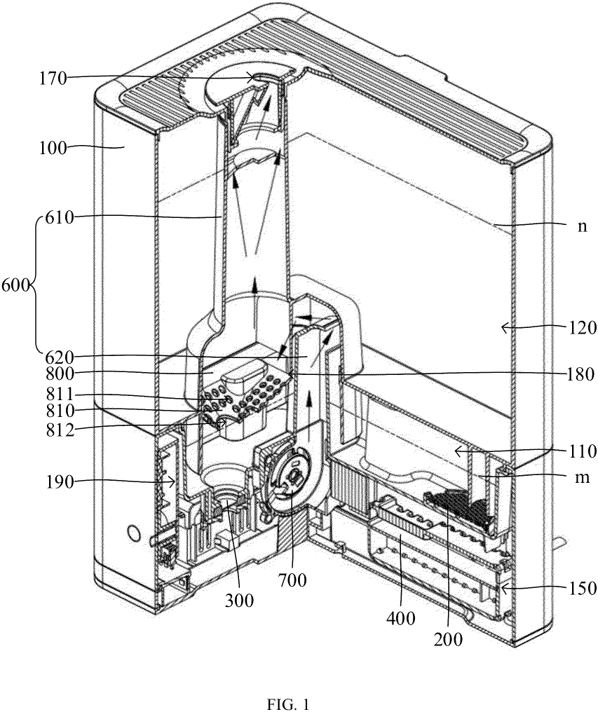

Referring to FIG. 1, the sterilization atomization device provided by an embodiment of the present application will now be described. The sterilization atomization device can be a humidifier, an aromatherapy atomizer, a fragrance diffuser, etc. and the sterilization atomization device is suitable for relatively closed spaces such as residential bedrooms, residential bathrooms, hospital wards, and shopping mall bathrooms, and the sterilization atomization device is suitable for relatively open spaces. The sterilization atomization device includes a housing 100, an electrolysis module 200, an atomization module 300, and a power module 400. The housing 100 is provided with a first liquid storage cavity 110, and the first liquid storage cavity 110 is configured for accommodating the electrolyte; the electrolysis module 200 is arranged in the first liquid storage cavity 110, and the electrolysis module 200 is configured for electrolysis of the electrolyte in the first liquid storage cavity 110; the atomization module 300 is arranged in the first liquid storage cavity 110, and the atomization module 300 is configured for atomizing the electrolyte in the first liquid storage cavity 110 into an atomized airflow, and the atomized airflow flows out of the housing 100 at least through the first liquid storage cavity 110; the power module 400 is configured for supplying power to electrolysis module 200 and atomization module 300 independently at the same time.

It should be noted that the electrolyte can be distilled water, tap water containing chlorine element, etc., and the electrolyte can be selected according to needs, which is not uniquely limited herein.

It should also be noted that the first liquid storage cavity 110can have various forms, for example, referring to FIG. 1, the first liquid storage cavity 110 can be a single cavity, and both the electrolysis module 200 and the atomization module 300 are arranged in the above-mentioned single cavity, the electrolysis module 200 can electrolyze the electrolyte in the above single cavity, and at the same time, the atomization module 300 can atomize the electrolyte in the above-mentioned single cavity. For another example, the first liquid storage cavity 110 can also include an electrolysis cavity and an atomization cavity that are partially isolated (which is not shown herein). It can be understood that the electrolysis cavity and the atomization cavity are not completely closed and isolated from each other. Instead, it is separated by relevant structures such as separators in some areas, and communicated with each other in other areas. The electrolyte in the electrolysis cavity can be injected into the atomization cavity through the unseparated area between the electrolysis cavity and the atomization cavity, the electrolysis module 200 is arranged in the above-mentioned electrolysis cavity, and is configured for electrolyzing the electrolyte in the electrolysis cavity, and the atomization module 300 is arranged in the above-mentioned atomization cavity, and is configured for atomizing the electrolyte injected from the electrolysis cavity into the atomization cavity. As a specific embodiment, the electrolysis cavity and the atomization cavity can be communicated only through an infusion tube provided with an infusion pump. The infusion pump can pump the electrolyzed electrolyte in the electrolysis cavity to the atomization cavity, and then the atomization module 300 atomizes the electrolyzed electrolyte and can also realize the sterilization function. In other embodiments, the first liquid storage cavity 110 can also take other forms, and can be arranged as required, which is not limited herein.

The sterilization atomization device provided by the present application, when in use, the electrolysis module 200 electrolyzes the electrolyte in the first liquid storage cavity 110, to generate strong oxidizing substances, such as, ground state oxygen O, ozoneO3, hydrogen peroxide H2O2, and hydroxide radicals —OH in the electrolyte, the above-mentioned strong oxidizing substances can plunder the electrons from microorganisms such as germs, so that the protein of the germs is strongly oxidized, thereby destroying the protein structure of the germs and killing germs in the electrolyte; at the same time, the atomization module 300 atomizes the electrolyte in the first liquid storage cavity 110 and forms an atomized airflow above the liquid level of the electrolyte, and the atomized airflow flows out to the outside of the housing 100.The atomized airflow can also kill germs in the air since the above-mentioned strong oxidizing substances contained therein, so that the sterilization atomization device provided in the present application can kill germs in the electrolyte and in the air at the same time, and the sterilization effect is good.

Compared with the related art, in the sterilization atomization device provided by the present application, the power module 400 can simultaneously supply power to the electrolysis module 200 and the atomization module 300 independently, that is, the power module 400 can simultaneously supply power for the electrolysis module 200 and atomization module 300, and the circuit where the electrolysis module 200 is located and the circuit where the atomization module 300 is located do not share the same ground. In this way, it can not only ensure that the electrolysis module 200 and the atomization module 300 can work at the same time, ensure the sterilization effect, but also ensure that when the electrodes of the electrolysis module 200 (such as the electrodes including the titanium metal layer and the platinum metal layer coated on the surface of the titanium metal layer) and the metal materials on the atomization module 300 (such as the silver paste layer for electrical connection with the power module 400, and other metal materials used as electrode of the atomization module 300) are exposed in the electrolyte, no electrolytic reaction will occur between the electrodes of the electrolysis module 200 and the metal materials on the atomization module 300,and the metal materials on the atomization module 300 will not be corroded, which is beneficial to improve the service life of the sterilization atomization device.

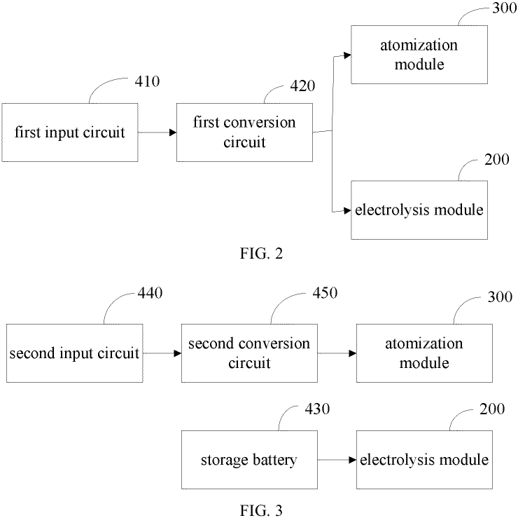

In another embodiment of the present application, referring to FIG. 2, the power module 400 includes a first input circuit 410 and a first conversion circuit 420 that are connected to each other, and the first conversion circuit 420 is configured for converting the first input voltage of the first input circuit 410 into a first output voltage and a second output voltage that are mutually isolated, the first output voltage is transmitted to the atomization module 300, and the second output voltage is transmitted to the electrolysis module 200.

It should be noted that, in general, the operating voltages of the electrolysis module 200 and the atomization module 300 are different, and the first conversion circuit 420 can use the circuit isolation principle to convert one input voltage into two mutually isolated output voltages. Those skilled in the art can arrange the first conversion circuit 420 according to the art, such that the first output voltage and the second output voltage can meet the power supply requirements of the atomization module 300 and the electrolysis module 200 respectively, and the specific structure of the first conversion circuit 420 is not described herein.

With the above structure, the circuit where the atomization module 300 is located and the circuit where the electrolysis module 200 is located are not share the same ground. When the metal materials on the atomization module 300 and the electrodes of the electrolysis module 200 are exposed to the electrolyte at the same time, the electrolysis reaction between the metal materials on the atomization module 300 and the electrodes of the electrolysis module 200 will not occur, which can avoid electrochemical corrosion of the metal materials on the atomization module 300, and is beneficial to improve the service life of the sterilization atomization device.

In an embodiment, the first input circuit 410 is a commercial power input circuit.

In another embodiment of the present application, referring to FIG. 3, the power module 400 includes a storage battery 430, a second input circuit 440 and a second conversion circuit 450 connected to each other, and the second conversion circuit 450 is configured for converting the second input voltage of the second input circuit 440into a third output voltage and transmitted the third output voltage to the atomization module 300, and the electrolysis module 200 is powered by the storage battery 430.

With the above structure, the electrolysis module 200 is powered by the storage battery 430, and the atomization module 300 is powered by the second input circuit 440 and the second conversion circuit 450,the circuit where the electrolysis module 200 is located and the circuit where the atomization module 300 is located are also not share the same ground. When the metal materials of the atomization module 300and the electrodes of the electrolysis module 200 are exposed to the electrolyte at the same time, no electrolytic reaction will occur between the electrodes of the electrolysis module 200 and the metal materials on the atomization module 300,which can avoid electrochemical corrosion of the metal material on the atomization module 300, and is beneficial to improve the service life of the sterilization atomization device.

In an embodiment, the second input circuit 440 is a commercial power input circuit.

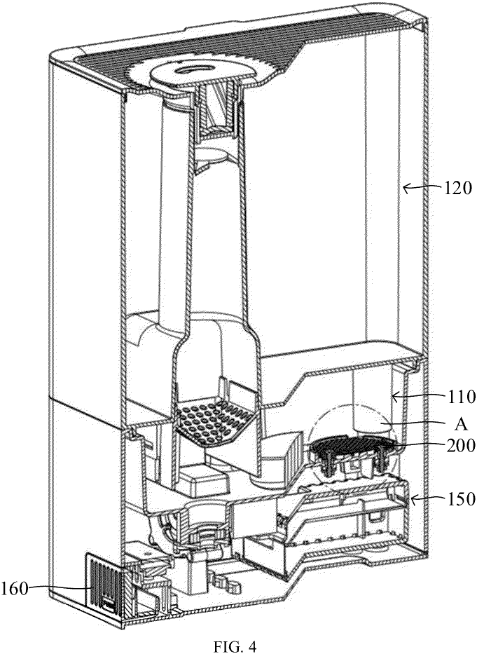

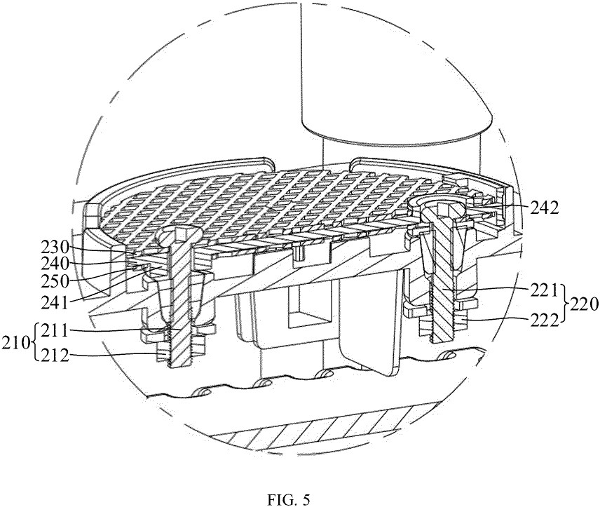

In another embodiment of the present application, referring to FIG. 4 and FIG. 5, the electrolysis module 200 includes a first connection assembly 210, a second connection assembly 220, a first electrode sheet 230, an insulating sheet 240, and a second electrode sheet 250.The first electrode sheet 230, the insulating sheet 240, and the second electrode sheet 250 are stacked in sequence on the cavity wall of the first liquid storage cavity 110 in a direction away from the cavity wall of the first liquid storage cavity 110 to approaching the cavity wall of the first liquid storage cavity 110.The first connection assembly 210 is configured for fixing the first electrode sheet 230 to the cavity wall of the first liquid storage cavity 110 and configured for connecting the first electrode sheet 230 to the power module 400,the second connection assembly 220 is configured for fixing the second electrode sheet 250 to the cavity wall of the first liquid storage cavity 110 and configured for connecting the second electrode sheet 250 to the power module 400.

With the above structure, the first electrode sheet 230 is fixed to the cavity wall of the first liquid storage cavity 110 through the first connection assembly 210 and is electrically connected to the power module 400,and the second electrode sheet 250 is fixed to the cavity wall of the first liquid storage cavity 110 through the second connection assembly 220and is electrically connected to the power module 400, and the insulating sheet 240 for isolating the first electrode sheet 230 and the second electrode sheet 250 is directly sandwiched between the first electrode sheet 230 and the second electrode sheet 250, and the mounting of the electrolysis module 200 is simple and convenient, the electrolyte in the first liquid storage cavity 110 can be electrolyzed to generate strong oxidizing substances with a sterilization function, so as to achieve the sterilization effect.

Specifically, the first connection assembly 210 includes a first bolt 211 and a first nut 212. The first bolt 211 extends the first electrode sheet 230, the insulating sheet 240, the second electrode sheet 250, and the cavity wall of the first liquid storage cavity 110 in sequence. The head portion of the first bolt 211 is pressed against the first electrode sheet 230 and is electrically connected to the first electrode sheet 230;and the region of the first bolt 211 corresponding to the second electrode sheet 250 is separated from the second electrode sheet 250 by the first convex ring 241 protruding toward the second electrode sheet 250 on the insulating sheet 240. The tail portion of the first bolt 211 protrudes out of the first liquid storage cavity 110 and is electrically connected to the power module 400,and the first nut 212 is arranged at the tail portion of the first bolt 211 for locking the first bolt 211 against the cavity wall of the first liquid storage cavity 110.

Specifically, the second connection assembly 220 includes a second bolt 221 and a second nut 222.The second bolt 221 extends the first electrode sheet 230, the insulating sheet 240, the second electrode sheet 250, and the cavity wall of the first liquid storage cavity 110 in sequence. The head portion of the second bolt 221is pressed against the second electrode sheet 250 and is electrically connected to the second electrode sheet 250; and the region of the second bolt 221 corresponding to the first electrode sheet 230 is separated from the first electrode sheet 230 by the second convex ring 242 protruding toward the first electrode sheet 230 on the insulating sheet 240. The tail portion of the second bolt 221 protrudes out of the first liquid storage cavity 110 and is electrically connected to the power module 400, and the second nut 222 is arranged at the tail portion of the second bolt 221 for locking the second bolt 221 against the cavity wall of the first liquid storage cavity 110.

In an embodiment, both the first electrode sheet 230 and the second electrode sheet 250 are grid electrode sheets.

In another embodiment of the present application, the atomization module 300 includes an ultrasonic atomization sheet, and the ultrasonic atomization sheet includes a piezoelectric ceramic sheet (not shown) and two silver paste layers respectively disposed on the upper surface and the lower surface of the piezoelectric ceramic sheet (not shown), and two silver paste layers are configured for electrical connection with power module 400.

With the above structure, the atomization module 300 utilizes electronic high-frequency oscillation, which can disperse the liquid electrolyte molecules to generate atomized airflow, and the atomized particles generated by the electronic high-frequency oscillation are relatively uniform in size, such that the air and the atomized particles are easier to fully contact to improve the sterilization effect.

In an embodiment, the atomization module 300 is further provided with a silicone waterproof cover (not shown), and the silicone waterproof cover can waterproof the silver paste layer on the side facing the first liquid storage cavity 110 to avoid exposure of the silver paste layer on the side to the electrolyte.

In another embodiment of the present application, at least one of the first electrode sheet 230 and the second electrode sheet 250 includes a titanium metal layer (not shown) and a composite coating layer (not shown) coated on the surface of the titanium metal layer. The composite coating layer can be a ruthenium-iridium composite material layer, a ruthenium-platinum composite material layer, or a platinum-iridium composite material layer. With the above structure, the electrolysis module 200 has higher electrolysis efficiency, and no electrolytic reaction occurs when each electrode sheet and the silver paste layer of the ultrasonic atomization sheet are simultaneously in the electrolyte.

It should also be noted that, when the electrolyte is an electrolyte with a tendency to form scale, in order to prevent calcium ions, magnesium ions, etc. in the electrolyte from combining with acid radicals to form scale, when using the sterilization atomization device provided by the present application, the polarity of the first electrode sheet 230 and the second electrode sheet 250 will be changed periodically. For example, when the sterilization atomization device starts to work, the first electrode sheet 230 is used as the positive electrode, and the second electrode sheet 250 is used as the negative electrode, after working for a preset time, such as after 35 seconds or 60 seconds, the first electrode sheet 230 is used as the negative electrode, and the second electrode sheet 250 is used as the positive electrode, and then the polarities of electrode sheets are cyclically changed in this way. In this mode, both the first electrode sheet 230 and the second electrode sheet 250 are needed to include the titanium metal layer and the composite coating layer applied to the surface of the titanium metal layer.

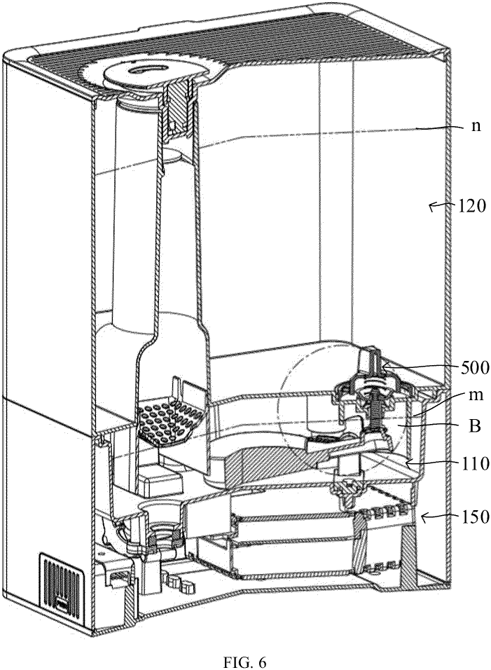

In another embodiment of the present application, referring to FIG. 6, the sterilization atomization device further includes a switch module 500, the housing 100 further provides a second liquid storage cavity 120 for accommodating the electrolyte, and the switch module 500 is configured for communicating the second liquid storage cavity120 and the first liquid storage cavity110 when a liquid level of the first liquid storage cavity110 is lower than a first preset value, such that the second liquid storage cavity 120 supplies the electrolyte to the first liquid storage cavity 110,and the switch module 500 is configured for isolating the second liquid storage cavity 120 from the first liquid storage cavity 110 when the liquid level of the first liquid storage cavity 110 is higher than a second preset value, such that the second liquid storage cavity 120 stops supplying the electrolyte to the first liquid storage cavity 110, and the first preset value is less than or equal to the second preset value.

It should be noted that, in general, when the atomization module 300 is an ultrasonic atomization sheet, the ultrasonic atomization sheet is energized when the electrolyte is insufficient, and the pulse current to start the operation may bum out the ultrasonic atomization sheet; when the depth of the electrolyte is too deep, the high-frequency oscillation effect of the ultrasonic atomization sheet is poor, and the atomization effect is easily worse. Therefore, the amount of the electrolyte in the first liquid storage cavity 110 needs to be suitable. Referring to FIG. 1 and FIG. 6,the liquid level of the electrolyte in the first liquid storage cavity 110 is suitable to be maintained at the first liquid level line m.

Specifically, in an embodiment, the switch module 500 includes a solenoid valve and a liquid level detector. The solenoid valve is configured for controlling the opening and closing of the second liquid storage cavity 120 and the first liquid storage cavity 110, and the liquid level detector is configured for detecting a liquid level height of the electrolyte. In other embodiments, the switch module 500 can further adopt other structures, which are not limited herein.

In the sterilization atomization device provided in the present embodiment, by arranging the first liquid storage cavity 110 and the second liquid storage cavity 120 isolated from each other, and the switch module 500.The liquid level of the electrolyte in the first liquid storage cavity 110 can be arranged between the first preset value and the second preset value, which is beneficial to the stable operation of the atomization module 300.

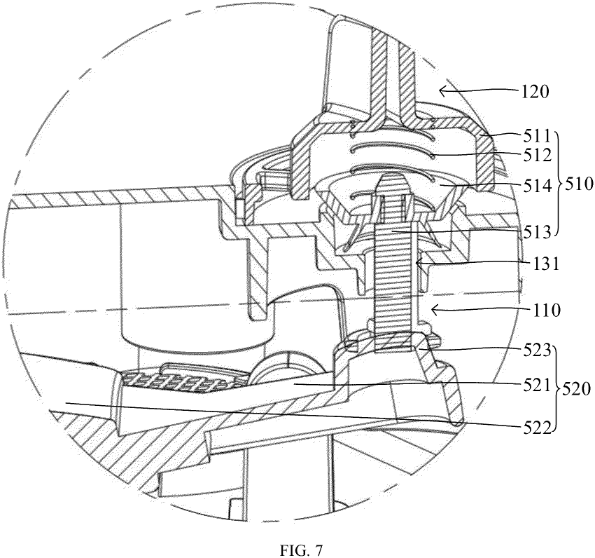

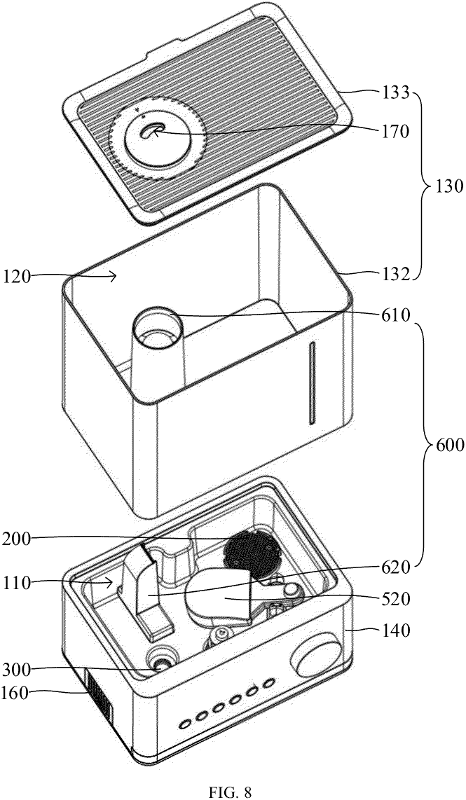

In another embodiment of the present application, referring to FIG. 7 and FIG. 8, the housing 100 includes a liquid storage tank 130 and a base 140, the liquid storage tank 130 is detachably mounted on the base 140, and second liquid storage cavity 120is provided in the liquid storage tank 130, the bottom portion of the liquid storage tank 130 and the top portion of the base 140 are enclosed to form the first liquid storage cavity 110,and the bottom portion of the liquid storage tank 130 is provided with a water outlet 131 that communicates the second liquid storage cavity 120 and the first liquid storage cavity 110.The first preset value is equal to the second preset value. The switch module 500 includes a normally shut switch 510 and a ball float type trigger 520. The normally shut switch 510 is arranged at the water outlet 131, and the ball float type trigger 520 is arranged on the top portion of the base 140 and is configured for triggering the normally shut switch 510 to open when the liquid level is lower than the first preset value.

With the above structure, after the liquid storage tank 130 is mounted on the base 140, the normally shut switch 510 and the ball float type trigger 520 cooperate to maintain the liquid level of the electrolyte in the first liquid storage cavity 110 at the first preset value, after the liquid storage tank 130 is detached from the base 140, the normally shut switch 510 closes the water outlet 131,so that the liquid storage tank 130 can be detached from the base 140 and the electrolyte can be filled into the second liquid storage cavity 120. Referring to FIG. 1 and FIG. 6, the liquid level of the electrolyte in the second liquid storage cavity 120 can be maintained at the second liquid level line n.

Specifically, referring to FIG. 7, the normally shut switch 510 includes a valve cover 511, an elastic member 512 and a valve rod 513. The cavity walls of the valve cover 511 and the second liquid storage cavity 120 are fixed at a side of water outlet 131 close to the second liquid storage cavity 120 at intervals. The valve rod 513 is extended in the water outlet 131, and a side of the valve rod 513 close to the second liquid storage cavity 120 is provided with a cover plate 514 capable of covering the water outlet 131, and the elastic member 512 is abutted between the valve cover 511 and the cover plate 514 of the valve rod 513 for pressing the cover plate 514against the water outlet 131, so that the cover plate 514 covers the water outlet 131, and one end of the valve rod 513 away from the second liquid storage cavity 120 extends into the first liquid storage cavity 110 adjacent to the second liquid storage cavity 120.The ball float type trigger 520 includes a connecting rod 521 and a ball float 522 and an ejector rod 523 which are respectively provided at both ends of the connecting rod 521. The middle portion of the connecting rod 521 is hinged on the cavity wall of the first liquid storage cavity 110, and the ejector rod 523 is located at a position corresponding to the valve rod 513 to push the valve rod 513 to move up and down. When the liquid level in the first liquid storage cavity 110 is lower than the first preset value, the ball float 522 floats on the liquid level of the electrolyte under the action of buoyancy, and the ejector rod 523 is in a position where it can touch the valve rod 513, and the cover plate 514 of the valve rod 513 is in a position that does not cover the water outlet 131,and the electrolyte in the second liquid storage cavity 120 can gradually flow from the water outlet 131 into the first liquid storage cavity 110 under the action of gravity; as the electrolyte gradually flows into the first liquid storage cavity 110, the liquid level of the electrolyte in the first liquid storage cavity 110 gradually rises, the ball float 522 rises continuously with the rise of the liquid level of the electrolyte, and the ejector rod 523 gradually moves downward under the driving action of the ball float 522, when the liquid level in the first liquid storage cavity 110 rises and reaches the first preset value, the ejector rod 523 moves downward to the position where it is separated from the valve rod 513. The cover plate 514 of the valve rod 513 covers the water outlet 131 under the action of the elastic member 512,the electrolyte in the second liquid storage cavity 120 stops flowing into the first liquid storage cavity 110,and the liquid level of the electrolyte in the first liquid storage cavity 110 is maintained at the first preset value, and when the electrolyte in the first liquid storage cavity 110 is gradually consumed and is lower than the first preset value, the above process will be repeated to maintain the electrolyte in the first liquid storage cavity 110 at the first preset value.

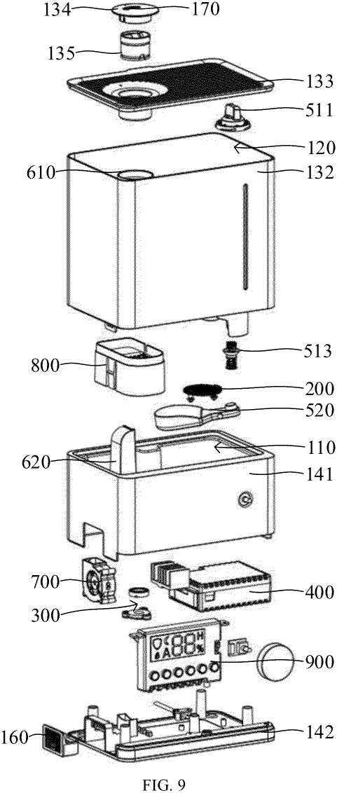

In an embodiment, referring to FIG. 8 and FIG. 9,the liquid storage tank 130 includes a liquid storage tank body 132 with an opening at the upper end and a tank cover 133 which is covered on the liquid storage tank body 132 and the opening, and the tank cover 133 can be opened to add the electrolyte to the liquid storage tank body 132.

In an embodiment, referring to FIG. 9, the tank cover 133 is provided with an air outlet 170 and an air outlet plate 134 and a fixing member 135 for fixing the air outlet plate 134.

In an embodiment, referring to FIG. 8 and FIG. 9,the base 140 includes a base body 141 with an opening at the bottom thereof and a bottom cover 142, and the bottom cover 142 is covered at the base body 141and the opening at the bottom. An air inlet cavity 150 described below is formed between the bottom cover 142 and the base body 141.

In another embodiment of the present application, referring to FIG. 1, the housing 100 further includes the air inlet cavity 150 and an air inlet cavity 160 communicating with the air inlet cavity 150 (referring to FIG. 4), and the air inlet cavity 150 is provided below the first liquid storage cavity 110.The sterilization atomization device further includes a mist guiding tube 600 and a fan module 700, the bottom portion of the mist guiding tube 600 is communicated with the air inlet cavity 150,the middle portion of the mist guiding tube 600 is communicated with the first liquid storage cavity 110, and the top portion of the mist guiding tube 600is communicated with the outside of the housing 100.The fan module 700 is located at the bottom portion of the mist guiding tube 600. The fan module 700 is configured for driving the external air to enter the mist guiding tube 600 through the air inlet 160 and the air inlet cavity 150 in turn, and drive the air entering the mist guiding tube 600 to carry the atomized airflow at the top portion of the first liquid storage cavity 110 to the outside of the housing 100. With the above structure, under the action of the fan module 700, the external air enters the inner cavity of the mist guiding tube 600 through the air inlet 160 and the air inlet cavity 150, and the air entering the inner cavity of the mist guiding tube 600 carries the atomized airflow at the top portion of the first liquid storage cavity 110 to the outside of the housing 100, which can humidify and sterilize the external environment.

In an embodiment, referring to FIG. 1 and FIG. 8,when the housing 100 includes the above-mentioned liquid storage tank 130 and the base 140,the bottom portion of the mist guiding tube 600 is communicated with the air inlet cavity 150, and the middle portion of the mist guiding tube 600is communicated with the first liquid storage cavity 110,and the mist guiding tube 600 is communicated with the outside after passing through the first liquid storage cavity 110 and the second liquid storage cavity 120 in sequence. Specifically, the liquid storage tank 130 is provided with an air outlet 170 that is communicated with the outside, and the top portion of the mist guiding tube 600 is communicated with the air outlet 170.

Specifically, referring to FIG. 1 and FIG. 8, the mist guiding tube 600 can include an upper guiding tube 610 and a lower guiding tube 620 which are communicated with each other, and the arrows inside the upper guiding tube 610 and the lower guiding tube 620 in FIG. 1 are represented as atomized airflow and the flow direction of the air in the upper guiding tube 610 and lower guiding tube 620.Both the upper guiding tube 610 and the lower guiding tube 620 are defined with open air channels at both ends. The upper guiding tube 610 is located on the bottom wall of the second liquid storage cavity 120, both ends of the upper guiding tube 610 are communicated with the air outlet 170 and the first liquid storage cavity 110 respectively, and the outer diameter of the upper guiding tube 610 gradually decreases along the direction from the bottom of the liquid storage tank 130 to the top of the liquid storage tank 130. That is, the cross-section of the bottom of the upper guiding tube 610 is larger than the cross-section of the top of the upper guiding tube 610.The bottom portion of the upper guiding tube 610 can be communicated with the air inlet cavity 150 through the lower guiding tube 620, and can also be communicated with the first liquid storage cavity 110through the noise reduction module 800. The lower guiding tube 620 is arranged at the bottom wall of the first liquid storage cavity 110, and the bottom portion of the lower guiding tube 620 protrudes into the air inlet cavity 150,and the top portion of the lower guiding tube 620 extends the first liquid storage cavity 110 and protrudes into the bottom portion of the upper guiding tube 610, and the lower guiding tube 620 and the upper guiding tube 610 are not on the same line, that is, the lower guiding tube 620 is not directly below the upper guiding tube 610, and the opening of an end of the lower guiding tube 620 extending into the upper guiding tube 610 is arranged in a horizontal direction. When the sterilization atomization device is working, the external air enters the bottom of the lower guiding tube 620 through the air inlet 160 and the air inlet cavity 150 in turn under the action of the fan module 700, and the external air flows upward in the vertical direction in the lower guiding tube 620, then the external air flows in the horizontal direction at the exit of the lower guiding tube 620 to the upper guiding tube 610, and then the external air flows in the horizontal direction at the bottom of the upper guiding tube 610 to the area corresponding to the atomization module 300 of the upper guiding tube 610, and finally the external air carries the atomized airflow above the first liquid storage cavity 110 and flows vertically upwards to the air outlet 170 in the upper guiding tube 610. With the structure, the upper guiding tube 610 and the lower guiding tube 620 are located on the liquid storage tank 130 and the base 140, respectively, and are formed separately, which is convenient to the manufacture of the mist guiding tube 600 and the sterilization atomization device.

In an embodiment, the power module 400 is arranged in the air inlet cavity 150.

In another embodiment of the present application, referring to FIG. 1, a region corresponding to the atomization module 300 on the top wall of the first liquid storage cavity 110 is provided with a mounting hole 180 that communicates with the mist guiding tube 600, and the sterilization atomization device further includes the noise reduction module 800, the noise reduction module 800 is mounted at the mounting hole 180.

In an embodiment, the noise reduction module 800 includes a drainage cover 810 that is flared from the bottom to the top of the first liquid storage cavity 110, and the drainage cover 810 is provided with a plurality of first through holes 811 and one second through hole 812, the second through hole 812 is surrounded by the plurality of first through holes 811, the cross-sectional area of the second through hole 812 is larger than that of the first through hole 811, and the second through hole 812 is located at the most bottom of the noise reduction module 800, that is, the second through hole 812 is arranged at the position on the noise reduction module 800 closest to the electrolyte within the first liquid storage cavity 110.

It should be noted that, during the atomization process of atomization module 300, especially the atomization process of the ultrasonic atomization sheet, the electrolyte stirred up by the atomization module 300 continuously spray upwards, and after hitting a certain height, the electrolyte then falls back to the bottom of the first liquid storage cavity 110. In the sterilization atomization device provided in the embodiment, the electrolyte falls back to the wall of the drainage cover 810 of the noise reduction module 800 during the process of spraying upward and then falling back down, and under the guidance of the drainage cover 810, the electrolyte flows to the liquid level of the electrolyte, which can reduce the impact of the sprayed electrolyte on the liquid level of the electrolyte, and reduce the noise when the sterilization atomization device is working.

In another embodiment of the present application, referring to FIG. 1 and FIG. 9, the sterilization atomization device further includes a control module 900, and the control module 900 is electrically connected to the electrolysis module 200, the atomization module 300, and the power module 400, respectively; the housing 100 is further includes a control cavity 190, and a control module 900 is located in the control cavity 190. The control module 900 can coordinate and control the working states of the electrolysis module 200, the atomization module 300, and the power module 400, so that the sterilization atomization device can automatically realize the functions of sterilization and humidification.

In an embodiment, the sterilization atomization device is provided with a fan module 700, and the fan module 700 is electrically connected with the control module 900.

In an embodiment, when the housing 100 includes the above-mentioned liquid storage tank 130 and the base 140, the control cavity 190 is provided in the base 140 and located outside the base 140, so that the operator can operate the control module 900 conveniently.

The aforementioned embodiments are only preferred embodiments of the present application, and should not be regarded as being limitation to the present application. Any modification, equivalent replacement, improvement, and so on, which are made within the spirit and the principle of the present application, should be included in the protection scope of the present application.

What is claimed is:

1. A sterilization atomization device, comprising:
a housing, provided with a first liquid storage cavity configured for accommodating an electrolyte;
an electrolysis module, disposed in the first liquid storage cavity and configured for electrolyzing the electrolyte accommodated in the first liquid storage cavity;
an atomization module, disposed in the first liquid storage cavity and configured for atomizing the electrolyte accommodated in the first liquid storage cavity into an atomized airflow, wherein the atomized airflow flows out of the housing through at least the first liquid storage cavity; and
a power module, configured for supplying power to the electrolysis module and the atomization module independently at the same time.

2. The sterilization atomization device according to claim 1, wherein the power module comprises a first input circuit and a first conversion circuit that are connected to each other, and the first conversion circuit is configured for converting a first input voltage of the first input circuit into a first output voltage and a second output voltage that are mutually isolated, and the first output voltage and the second output voltage are respectively transmitted to the atomization module and the electrolysis module.

3. The sterilization atomization device according to claim 1, wherein the power module comprises a storage battery, a second input circuit and a second conversion circuit, and the second conversion circuit is configured for converting a second input voltage of the second input circuit into a third output voltage, and the third output voltage is transmitted to the atomization module, and the electrolysis module is powered by the storage battery.

4. The sterilization atomization device according to claim 1, wherein the electrolysis module comprises a first connection assembly, a second connection assembly, a first electrode sheet, an insulating sheet and, a second electrode sheet; the first electrode sheet, the insulating sheet and the second electrode sheet are stacked in sequence, the first connection assembly is configured for fixing the first electrode sheet on a cavity wall of the first liquid storage cavity and for connecting the first electrode sheet to the power module; and the second connection assembly is configured for fixing the second electrode sheet on the cavity wall of the first liquid storage cavity and for connecting the second electrode sheet to the power module.

5. The sterilization atomization device according to claim 4, wherein at least one of the first electrode sheet and the second electrode sheet comprises a titanium metal layer and a composite coating layer coated on a surface of the titanium metal layer, and the composite coating layer is a ruthenium-iridium composite material layer, a ruthenium-platinum composite material layer or a platinum-iridium composite material layer.

6. The sterilization atomization device according to claim 1, wherein the atomization module comprises a piezoelectric ceramic sheet and two silver paste layers respectively disposed on an upper surface and a lower surface of the piezoelectric ceramic sheet, and the two silver paste layers are configured for electrically connecting with the power module.

7. The sterilization atomization device according to claim 1, wherein the sterilization atomization device further comprises a switch module, and the housing is further provided with a second liquid storage cavity configured for accommodating the electrolyte, the switch module configured for communicating the second liquid storage cavity and the first liquid storage cavity when a liquid level of the first liquid storage cavity is lower than a first preset value, such that the second liquid storage cavity supplies the electrolyte to the first liquid storage cavity, and the switch module is configured for isolating the second liquid storage cavity from the first liquid storage cavity when the liquid level of the first liquid storage cavity is higher than a second preset value, such that the second liquid storage cavity stops supplying the electrolyte to the first liquid storage cavity, and the first preset value is less than or equal to the second preset value.

8. The sterilization atomization device according to claim 7, wherein the housing comprises a liquid storage tank and a base, the liquid storage tank is detachably mounted on the base, the second liquid storage cavity is arranged in the liquid storage tank, and a bottom portion of the liquid storage tank and a top portion of the base are enclosed to form the first liquid storage cavity, the bottom portion of the liquid storage tank is provided with a water outlet connecting the second liquid storage cavity and the first liquid storage cavity, the first preset value is equal to the second preset value, and the switch module comprises a normally shut switch and a ball float type trigger, the normally shut switch is arranged at the water outlet, and the ball float type trigger is arranged at the top portion of the base and is configured for triggering the normally shut switch to open when the liquid level is lower than the first preset value.

9. The sterilization atomization device according to claim 1, wherein the housing further comprises an air inlet cavity and an air inlet communicated with the air inlet cavity, and the air inlet cavity is arranged below the first liquid storage cavity; the sterilization atomization device further comprises a mist guiding tube and a fan module, a bottom portion of the mist guiding tube is communicated with the air inlet cavity, a middle portion of the mist guiding tube is communicated with a top portion of the first liquid storage cavity, and a top portion of the mist guiding tube is communicated with an outside of the housing; the fan module is arranged at the bottom portion of the mist guiding tube, and the fan module is configured for driving an air entering the mist guiding tube through the air inlet and the air inlet cavity in turn to carry the atomized airflow from the top portion of the first liquid storage cavity to an outside of the housing.

10. The sterilization atomization device according to claim 9, wherein a region corresponding to the atomization module on a top wall of the first liquid storage cavity is provided with a mounting hole configured for communicating with the middle portion of the mist guiding tube, the sterilization atomization device further comprises a noise reduction module, and the noise reduction module is mounted at the mounting hole.

11. The sterilization atomization device according to claim 1, wherein the sterilization atomization device further comprises a control module, the control module is electrically connected with the electrolysis module and the atomization modules, respectively; the housing further includes a control cavity, and the control module is arranged in the control cavity.

12. The sterilization atomization device according to claim 2, wherein the sterilization atomization device further comprises a control module, the control module is electrically connected with the electrolysis module and the atomization modules, respectively; the housing further includes a control cavity, and the control module is arranged in the control cavity.

13. The sterilization atomization device according to claim 3, wherein the sterilization atomization device further comprises a control module, the control module is electrically connected with the electrolysis module and the atomization modules, respectively; the housing further includes a control cavity, and the control module is arranged in the control cavity.

14. The sterilization atomization device according to claim 4, wherein the sterilization atomization device further comprises a control module, the control module is electrically connected with the electrolysis module and the atomization modules, respectively; the housing further includes a control cavity, and the control module is arranged in the control cavity.

15. The sterilization atomization device according to claim 5, wherein the sterilization atomization device further comprises a control module, the control module is electrically connected with the electrolysis module and the atomization modules, respectively; the housing further includes a control cavity, and the control module is arranged in the control cavity.

16. The sterilization atomization device according to claim 6, wherein the sterilization atomization device further comprises a control module, the control module is electrically connected with the electrolysis module and the atomization modules, respectively; the housing further includes a control cavity, and the control module is arranged in the control cavity.

17. The sterilization atomization device according to claim 7, wherein the sterilization atomization device further comprises a control module, the control module is electrically connected with the electrolysis module and the atomization modules, respectively; the housing further includes a control cavity, and the control module is arranged in the control cavity.

18. The sterilization atomization device according to claim 8, wherein the sterilization atomization device further comprises a control module, the control module is electrically connected with the electrolysis module and the atomization modules, respectively; the housing further includes a control cavity, and the control module is arranged in the control cavity.

19. The sterilization atomization device according to claim 9, wherein the sterilization atomization device further comprises a control module, the control module is electrically connected with the electrolysis module and the atomization modules, respectively; the housing further includes a control cavity, and the control module is arranged in the control cavity.

20. The sterilization atomization device according to claim 10, wherein the sterilization atomization device further comprises a control module, the control module is electrically connected with the electrolysis module and the atomization modules, respectively; the housing further includes a control cavity, and the control module is arranged in the control cavity.
